(19) United States
(12) Patent Application Publication
(10) Pub. No.: US 2024/0054575 A1
(43) Pub. Date: Feb. 15, 2024

(54) METHOD FOR OPTIMISATION OF THE SUSTAINABILITY FOOTPRINT OF POLYMER FORMULATIONS

(71) Applicant: SABIC Global Technologies B.V., Bergen op Zoom (CN)

(72) Inventors: Maria Soliman, Selfkant (DE); Teena Bonizzi, Maastricht (NL); Johan Pastwa, Elsloo (NL); Mahari Tjahjadi, Shanghai (CN)

(21) Appl. No.: 18/268,874

(22) PCT Filed: Dec. 20, 2021
(86) PCT No.: PCT/EP2021/086716

§ 371 Date: Jun. 21, 2023

(30) Foreign Application Priority Data
Dec. 22, 2020 (EP) 20216611.2

(51) Int. Cl. G06Q 50/04; G06Q 50/26; C08L 23/06

(52) U.S. Cl. CPC G06Q 50/04; G06Q 50/26; C08L 23/06; C08L 2207/066; C08L 2207/20

(57) ABSTRACT

The present invention relates to a method for optimisation of the sustainability footprint of a polymer formulation, wherein the method involves: a. identifying one or more material specifications which are to be present in the polymer formulation; b. identifying one of more sustainability criteria to be optimised in the polymer formulation; c. providing a repository of materials that may be selected for use in the polymer formulation; d. providing a computer-implemented algorithm for calculating the contribution of each of the materials selected from the repository to achieving the desired material specifications of the polymer formulation; e. providing a computer-implemented algorithm for calculating the contribution of each of the materials selected from the repository to optimising the desired sustainability criteria of the polymer formulation; and f. calculating the composition of the polymer formulation wherein the polymer formulation demonstrates the optimal values for the sustainability criteria whilst meeting the material specifications.

The present invention relates to a polymer composition comprising recycled polyolefins and/or engineering thermoplastics.

In view of the current and foreseen increase in demand for circular use of materials, supporting reduction of material footprint, from the viewpoint of energy requirements, carbon emissions, fossil raw materials depletion as well as environmental pollution, the materials industry continues to seek developments in improvement of this circularity of its materials offerings.

Various materials solutions, also in the field of polyolefin and engineering thermoplastic materials, are currently offered by various parties in the value chain. These solutions typically include the use of a certain fraction of recycled materials. However, the requirements of e.g. quality, appearance, and processability that are imposed by the application for which a material is intended often pose limitations on the use of recycled materials in a materials solution for such application. There are various reasons as a result of which the use of recycled material is limited, including variations in the composition of the stream of recycled material that is supplied, contaminations present in the recycled material, variations in colour, and degradation of the material due to the fact that the material is subjected to multiple thermal processing steps.

To overcome these limitations, materials suppliers typically need to provide solutions in which the recycled material only forms part of the composition, and is complemented with high-quality polymer material that is produced according to conventional processes based on conventional, fossil feedstocks.

By so, the circularity of the material comprising the recycle polymer material is compromised in that only the fraction of such material composition that originates from a recycle stream can be understood to have a circular use, and the remainder fraction, often constituting a very significant fraction of the material composition, such as for example 75%, in fact demonstrates the very same footprint as any conventional, fossil-based polymer material; only additionally, processing steps such as a compounding step via melt extrusion need to be performed, also contributing to the energy and carbon footprint of such composition in a detrimental way.

Accordingly, there remains a continued need to increase the fraction of material in a polymer composition, particularly a polyolefin composition, that finds its origination in a polymer that is recycled from a previous consumer application.

In particular, there remains a need to produce a polyolefin composition having a [specify desirable material properties], wherein the quantity of used recycle materials is maximised.

The current inventors have developed a method for such optimisation of the sustainability footprint of a polymer formulation, wherein the method involves:
a. identifying one or more material specifications which are to be present in the polymer formulation;
b. identifying one of more sustainability criteria to be optimised in the polymer formulation;
c. providing a repository of materials that may be selected for use in the polymer formulation;
d. providing a computer-implemented algorithm for calculating the contribution of each of the materials selected from the repository to achieving the desired material specifications of the polymer formulation;
e. providing a computer-implemented algorithm for calculating the contribution of each of the materials selected from the repository to optimising the desired sustainability criteria of the polymer formulation; and
f. calculating the composition of the polymer formulation wherein the polymer formulation demonstrates the optimal values for the sustainability criteria whilst meeting the material specifications.

Such method, which commonly involves the application of a multi-factorial computation of parameters that jointly attribute to obtaining the desirable composition of matter, allows for arriving at the optimum composition to achieve the material specifications as well as the sustainability specifications. In particular, it allows for arriving at such composition without elaborate need for conducting experimental research, thereby being able to do so swiftly and economically.

In a suitable embodiment, the method is performed on a computer. For example, in the method, the repository of materials may be a database stored on a computer,

When striving to use waste streams of polymer materials, one typically is confronted with availability of streams or batches of material that may significantly fluctuate in their composition. A batch of waste polymer material, in particular waste that is collected after consumer use, commonly referred to as post-consumer recycle plastic waste or PCR, typically contains a large variety of plastic materials; one element of waste that ends up in the PCR stream may be very different in nature versus the next element; to give an example, a shampoo bottle typically is made out of a polymer material that is completely different from the polymer material of which a vegetables packaging is made, and again completely different from the polymer material of which a drinks bottle may be made. But all these, and more, end up together in PCR streams, and typically in different ratios. To make matters yet even further complicated, the polymer material of the same type that may be used in the same type of packaging may comprise quite different ingredients depending on from which manufacturer the polymer materials originate; a polyethylene composition from the first producer can be quite different in nature from one of a second producer. Even, ingredients incorporated into a polymer formulation by different producers to enable the same property to be achieved may, when combined into a recycled formulation, act counteractive vis-à-vis that property.

So, from the above, one will understand that the use of recycled plastics presents quite daunting challenges when one wants to deliver a product that is of consistent high quality, and that can be provided in large quantities.

In the field of processing of waste plastic streams such as PCR streams, certain techniques exist, and are under ongoing further development, to separate the supplied waste streams into certain streams of more consistent nature. Such separation processes, which may involve certain automated sorting methods as well as under certain circumstances manual sorting methods, may result in certain streams that are quite stable in composition, and may be fairly suitable for conversion into recycled polymer materials of certain specification via mechanical recycling methods, as well as in certain streams that are of more varying composition, and may be of less suitable quality for mechanical recycling.

In order to render the latter category of waste plastics streams of lesser quality and more variable composition suitable for use as polymer material in applications of certain demanding nature, conversion methods other than direct mechanical recycling are available and also under ongoing development. Such conversion methods may for example involve certain chemical recycling processes, in which the waste streams via chemical processes can be converted into building blocks for polymers of equal quality and purity as those currently used to manufacture polymers via conventional, and typically fossil-based, polymer manufacturing processes.

Using such polymer building blocks, typically also referred to as monomers, via chemical conversion processes based on waste polymer streams allows for manufacturing of a polymer that is of exactly the same quality as the conventional polymer that is based on conventional raw materials. Therefore, the properties of the polymer material or composition in which such polymer is used are as good as that of the conventionally used materials. The advantage of the use of such material obviously is related to the improved carbon and material footprint. This presents a particularly desirable opportunity to produce applications of the highest quality

Still, the route of using waste plastics via mechanical recycling may provide an option wherein the footprint in terms of energy, carbon and/or materials consumption is more attractive than would be for a conversion of waste plastics via chemical conversion routes.

As can be understood from the above, the process of arriving at a polymer composition having certain desirable, specified product properties whilst ensuring most optimal carbon, energy and materials footprint is a very complicated process, wherein the present method provides an invaluable support to achieving that result.

Since polyolefins form the ubiquitous polymer material in many areas of application, particularly in the field on non-durable applications like for example many packaging applications. Such plastic packaging material is typically being disposed of after a single use, and due to increasingly established processes of waste plastics collection available in the PCR streams, typically forming a major fraction of the plastics in the PCR streams. Therefore, the present invention particularly aims at providing a method for utilisation of waste polyolefins streams.

In a certain particular embodiment of the invention, the method encompasses that the polymer formulation comprises one or more mechanically recycled, preferably post-consumer mechanically recycled, polymer composition as material selected from the repository of materials. Such would benefit the sustainability footprint of the polymer formulation.

In a further particular embodiment, the method of the invention encompasses that the polymer formulation comprises one or more chemically recycled, preferably post-consumer chemically recycled, polymer material as material selected from the repository of materials. This also would provide benefit to the sustainability footprint of the polymer formulation. A particular advantage of polymer materials and compositions that are obtained by processes of chemical recycling of waste polymer materials is that such allows for processing of polymer waste fractions that in their nature are not suitable for direct application in their polymer form in the manufacturing of new articles having required and desirable product quality. This may be due to certain compositions of waste polymers containing polymeric products that have been subject to degradation during their lifespan. Also, this may be due to large variations of polymeric material constituents in the waste plastics composition, by means of which a direct thermoplastic conversion into a new and useable product, such as for example by thermal melt extrusion processes, injection moulding processes or thermoforming processes, is not possible. In such case, conversion of waste polymer streams via chemical recycling, which involves decomposition of the polymeric materials into suitable chemical building blocks, isolation of the various building blocks that result from such decomposition, and production of desired polymer materials from such isolated streams of chemical building blocks, allows for the production of high-quality polymers of the same specification and quality as would they have been produced from chemical building blocks obtained via conventional sources such as fossil hydrocarbon streams, whilst providing a certain sustainability benefit that originates from that fact that the feed materials used in the production of these polymers are waste polymer materials.

Particularly, a desirable embodiment of the invention related to a method wherein the polymer formulation comprises one or more mechanically recycled polymer composition, preferably a post-consumer mechanically recycled polymer composition, as well as one or more chemically recycled polymer material, preferably a post-consumer chemically recycled polymer material. In such case, the formulation provides both an advantageous benefit in terms of sustainability footprint from the mechanically recycled polymer composition, as well as the effect from the chemically recycled polymer material to make up for the desired material specifications that the formulation needs to deliver, which typically have been deteriorated in the mechanically recycled polymer composition due to its first use, in combination with the benefit in sustainability footprint that is additionally provided by the chemically recycled polymer material.

Particularly preferable is an embodiment wherein all polymer materials that are comprised in the polymer formulation are either mechanically recycled polymer compositions selected from the repository of materials, chemically recycled polymer materials selected from the repository of materials, or a combination thereof. In such situation, all polymer materials in the polymer formulation would provide a certain sustainability benefit, vis-à-vis a polymer formulation comprising polymer materials based on conventional, typically fossil-based, chemical building blocks.

The sustainability criteria which can be employed in the method of the invention to enable the algorithm to calculate the optimal polymer formulation may for example be selected from the list consisting of the energy consumption in manufacturing of the polymer formulation, the CO₂ emission in manufacturing of the polymer formulation, the quantity of fossil feedstock used as raw materials in the manufacturing of the polymer formulation, the quantity of fossil fuel-based energy used in the manufacturing of the polymer formulation, and the quantity of energy consumed in transport of the feedstocks used in manufacturing of the polymer formulation.

The material specifications that can be employed in the method of the invention to enable the algorithm to calculate the optimal polymer formulation may for example be selected from the list consisting of molecular weight distribution, copolymer distribution, pressure resistance, creep performance, film seal strength, film stretchability, film shrinkage behaviour, film puncture resistance, film tear strength, impact strength, stress crack resistance, haze, gloss, transparency, scratch resistance, tribological properties, surface roughness, UV resistance, chemical resistance, organoleptic properties, melt mass flow rate, density, flexural properties, tensile properties, as well as standard deviation for each of these specifications. For example, the impact strength may be the Izod notched or unnotched impact strength as measured in accordance with ISO 180, method A, of 2019, at 23° C. and/or at −30° C., or the Charpy impact strength as measured in accordance with ISO 179-1 of 2010, at 23° C. and/or at −30° C. For example, the stress crack resistance may be the ESCR as determined in accordance with ASTM D1693-15e1. For example, the melt mass-flow rate may be determined in accordance with ASTM D1238 of 2013, at 190° C. for polyethylene materials, or at 230° C. for polypropylene materials, using a load of 2.16 kg, 5 kg, 10 kg, or 21.6 kg. For example, the flexural properties may be the flexural modulus and/or the flexural strength as determined in accordance with ISO 178 of 2019. For example, the tensile properties may be the elastic modulus, the stress at yield, the stress at break, the strain at yield, and/or the strain at break, as determined in accordance with ISO 527-1 of 2012.

The polymer formulation may for example be a polyethylene-based polymer formulation. Preferably, the polymer formulation comprises >50.0 wt %, more preferably >75.0 wt %, even more preferably >90.0 wt % of polyethylenes, with regard to the total weight of polymer materials in the polymer formulation. Particularly preferably, the total quantity of polymer materials in the polymer formulation consists of polyethylenes.

The polymer formulation may comprise a single polyethylene-type material, or may comprise multiple polyethylene-type materials. For example, the polymer formulation may comprise at least two different polyethylene-type materials, preferably at least three different polyethylene-type materials. A polyethylene-type material as used in the context of this invention may also be understood as a polyethylene grade.

In the context of the present application, suitable polyethylene materials may be low-density polyethylenes (LDPE), linear low-density polyethylenes (LLDPE), or high-density polyethylenes (HDPE). It is preferred that the mechanically recycled polymer composition as used in the method of the present invention comprises ≥70.0 wt % of HDPE, LDPE and/or LLDPE, with regard to the total weight of the composition.

For example, the HDPE may have a density of ≥940 and ≤975 kg/m³, preferably of ≥945 and ≤970 kg/m³, more preferably of ≥950 and ≤965 kg/m³. For example, the LDPE may have a density of ≥900 and ≤935 kg/m³, preferably of ≥910 and ≤930 kg/m³, more preferably of ≥915 and ≤930 kg/m³. For example, the LLDPE may have a density of ≥850 and ≤935 kg/m³, preferably of ≥870 and ≤925 kg/m³, more preferably of ≥900 and ≤925 kg/m³. In the context of the present invention, the densities of the polyethylenes are determined in accordance with ASTM D792 (2008).

It is further preferred that each of the LDPE, HDPE and LLDPE has a melt mass-flow rate of ≥0.1 and ≤10.0 g/10 min, preferably of ≥0.1 and ≤5.0 g/10 min, more preferably of ≥0.1 and ≤2.0 g/10 min, as determined in accordance with ASTM D1238 (2013) at a temperature of 190° C., under a load of 2.16 kg.

It is further preferred that the chemically recycled material is high-density polyethylene (HDPE), low-density polyethylene (LDPE), or linear low-density polyethylene (LLDPE).

The polymer formulation may for example comprise ≥10.0 and ≤90.0 wt %, preferably ≥20.0 and ≤80.0 wt %, more preferably ≥30.0 and ≤70.0 wt %, of the post-consumer mechanically recycled polymer composition and/or ≥10.0 and ≤90.0 wt %, preferably ≥20.0 and ≤80.0 wt %, more preferably ≥30.0 and ≤70.0 wt %, of the post-consumer chemically recycled polymer material, with regard to the total weight of the polymer formulation.

The polymer formulation according to the present invention may for example be manufactured by melt extrusion, or by powder mixing.

The polymer formulation may for example be a polypropylene-based polymer formulation. Preferably, the polymer formulation comprises >50.0 wt %, more preferably >75.0 wt %, even more preferably >90.0 wt % of polypropylenes, with regard to the total weight of polymer materials in the polymer formulation. Particularly preferably, the total quantity of polymer materials in the polymer formulation consists of polypropylenes.

The polymer formulation may for example be a polycarbonate-based polymer formulation. Preferably, the polymer formulation comprises >50.0 wt %, more preferably >75.0 wt %, even more preferably >90.0 wt % of polycarbonates, with regard to the total weight of polymer materials in the polymer formulation. Particularly preferably, the total quantity of polymer materials in the polymer formulation consists of polycarbonates.

The polymer formulation may for example be a polyamide-based polymer formulation. Preferably, the polymer formulation comprises >50.0 wt %, more preferably >75.0 wt %, even more preferably >90.0 wt % of polyamides, with regard to the total weight of polymer materials in the polymer formulation. Particularly preferably, the total quantity of polymer materials in the polymer formulation consists of polyamides. The polyamides may for example be polyamide-6, polyamide-6,6, polyamide-12, polyamide-4,6, or any other polyamide produced using an aliphatic or aromatic dicarboxylic acid and an aromatic or aliphatic diamine. The polymer formulation may comprise one single polyamide or may comprise a mixture of different polyamides.

The polymer formulation may for example be a thermoplastic polyester-based polymer formulation. Preferably, the polymer formulation comprises >50.0 wt %, more preferably >75.0 wt %, even more preferably >90.0 wt % of thermoplastic polyesters, with regard to the total weight of polymer materials in the polymer formulation. Particularly preferably, the total quantity of polymer materials in the polymer formulation consists of thermoplastics polyesters. The thermoplastic polyesters may for example be polyethylene terephthalate, polybutylene terephthalate, or polyethylene furanoate. The polymer formulation may comprise one single thermoplastic polyester or may comprise a mixture of different thermoplastic polyesters.

The present invention, in a certain embodiment, also relates to a polymer formulation obtained according to the method of the invention.

The computer-implemented algorithm for calculating the contribution of the selected materials to achieving the materials specification and the sustainability criteria optimisation may for example comprise transfer functions that describes the implication of the choice of the material on the various material specifications and sustainability parameters. By ensuring that for each of the materials the transfer functions are included in the algorithms, the algorithms can deliver a desirable formulation as output, where the material specifications and sustainability specifications are provided to the algorithms as input.

In certain embodiments, the invention may also relate to a system wherein the method is implemented on a computer device, wherein the materials specifications and the sustainability specifications are supplied to the computer device as input parameters for the algorithms, and wherein the polymer formulations is provided as output.

In certain further specific embodiments, the system may comprise a production unit for producing the polymer composition that is connected to the computer device, wherein the output of the computer device provides a signal to the production unit that steers the composition of materials that are supplied to the production unit. This may for example be performed by steering supply speeds of individual material supply units each containing a dedicated material as provided for in the repository.

1. Method for optimisation of the sustainability footprint of a polymer formulation, wherein the method involves:
a. identifying one or more material specifications which are to be present in the polymer formulation;
b. identifying one of more sustainability criteria to be optimised in the polymer formulation;
c. providing a repository of materials that may be selected for use in the polymer formulation;
d. providing a computer-implemented algorithm for calculating the contribution of each of the materials selected from the repository to achieving the desired material specifications of the polymer formulation;
e. providing a computer-implemented algorithm for calculating the contribution of each of the materials selected from the repository to optimising the desired sustainability criteria of the polymer formulation; and
f. calculating the composition of the polymer formulation wherein the polymer formulation demonstrates the optimal values for the sustainability criteria whilst meeting the material specifications.

2. Method according to claim 1, wherein the method involves providing a computer on which the materials repository and the algorithms are implemented, and feeding the desired material specifications and sustainability criteria specification to the computer, based on which the computer calculates the polymer formulation according to the algorithms as output.

3. Method according to claim 1, wherein the polymer formulation comprises one or more post-consumer mechanically recycled polymer composition as material selected from the repository of materials.

4. Method according to claim 1, wherein the polymer formulation comprises one or more post-consumer chemically recycled polymer material as material selected from the repository of materials.

5. Method according to claim 1, wherein the sustainability criteria are selected from the list consisting of the energy consumption in manufacturing of the polymer formulation, the CO2 emission in manufacturing of the polymer formulation, the quantity of fossil feedstock used as raw materials in the manufacturing of the polymer formulation, the quantity of fossil fuel-based energy used in the manufacturing of the polymer formulation, and the quantity of energy consumed in transport of the feedstocks used in manufacturing of the polymer formulation.

6. Method according to claim 1, wherein the material specifications are selected from molecular weight distribution, copolymer distribution, pressure resistance, creep performance, film seal strength, film stretchability, film shrinkage behaviour, film puncture resistance, film tear strength, impact strength, stress crack resistance, haze, gloss, transparency, scratch resistance, tribological properties, surface roughness, UV resistance, chemical resistance, organoleptic properties, melt mass flow rate, density, flexural properties, tensile properties, and standard deviations for each of these specifications.

7. Method according to claim 1, wherein the polymer formulation is:
a polyethylene-based polymer formulation;
a polypropylene-based polymer formulation;
a polycarbonate-based polymer formulation;
a polyamide-based polymer formulation; or
a thermoplastic polyester-based formulation;
with regard to the total weight of the polymer formulation.

8. Method according to claim 1, wherein the polymer formulation comprises at least two different polyethylene-type materials.

9. Method according to claim 3, wherein the post-consumer mechanically recycled polymer composition comprises ≥70.0 wt % of high-density polyethylene (HDPE), low-density polyethylene (LDPE), and/or linear low-density polyethylene (LLDPE), with regard to the total weight of the composition, preferably wherein
the HDPE has a density of ≥940 and ≤975 kg/m3;
the LDPE has a density of ≥900 and ≤935 kg/m3; and
the LLDPE has a density of ≥850 and ≤935 kg/m3
as determined in accordance with ASTM D792 (2008).

10. Method according to claim 9, wherein each of the HDPE, the LDPE and the LLDPE has a melt mass-flow rate (MFR) of ≥0.1 and ≤2.0 g/10 min, as determined in accordance with ASTM D1238 (2013) at a temperature of 190° C. under a load of 2.16 kg.

11. Method according to claim 4, wherein the post-consumer chemically recycled material is high-density polyethylene (HDPE), low-density polyethylene (LDPE), or linear low-density polyethylene (LLDPE).

12. Method according to claim 4, wherein the polymer formulation comprises ≥10.0 and ≤90.0 wt % of the post-consumer mechanically recycled polymer composition and/or ≥10.0 and ≤90.0 wt % of the post-consumer chemically recycled polymer material, with regard to the total weight of the polymer formulation.

13. Method according to claim 1, wherein the polymer formulation is manufactured according to the calculated composition by melt extrusion mixing or by powder mixing.

14. Polymer formulation obtained according to the method of claim 1.

15. System comprising a computer device and a production unit for producing a polymer composition, wherein the computer device is operated according to the method of claim 1, wherein the computer device is connected to the production unit in such way that the output of the computer device is used as control input for the production unit.

16. System according to claim 15, wherein the output of the computer device is a signal or a set of signals that steer the composition of materials that are supplied to the production unit.

17. Method according to claim 1, wherein the polymer formulation is:
a polyethylene-based polymer formulation, comprising >90.0 wt % of polyethylenes;
a polypropylene-based polymer formulation, comprising >90.0 wt % of polypropylenes;
a polycarbonate-based polymer formulation, comprising >90.0 wt % of polycarbonates;
a polyamide-based polymer formulation, comprising >90.0 wt % of polyamides; or
a thermoplastic polyester-based formulation, comprising >90.0 wt % of thermoplastic polyesters;
with regard to the total weight of the polymer formulation.

18. Method according to claim 3, wherein the post-consumer mechanically recycled polymer composition comprises ≥70.0 wt % of high-density polyethylene (HDPE), low-density polyethylene (LDPE), and/or linear low-density polyethylene (LLDPE), with regard to the total weight of the composition, and wherein:
the HDPE has a density of ≥940 and ≤975 kg/m3;
the LDPE has a density of ≥900 and ≤935 kg/m3; and
the LLDPE has a density of ≥850 and ≤935 kg/m3
as determined in accordance with ASTM D792 (2008).

19. Method according to claim 4, wherein the post-consumer chemically recycled material is high-density polyethylene (HDPE), low-density polyethylene (LDPE), or linear low-density polyethylene (LLDPE), and wherein:
the HDPE has a density of ≥940 and ≤975 kg/m3;
the LDPE has a density of ≥900 and ≤935 kg/m3; and
the LLDPE has a density of ≥850 and ≤935 kg/m3
as determined in accordance with ASTM D792 (2008); and
wherein each of the HDPE, the LDPE and the LLDPE has a melt mass-flow rate (MFR) of ≥0.1 and ≤2.0 g/10 min, as determined in accordance with ASTM D1238 (2013) at a temperature of 190° C. under a load of 2.16 kg.

20. System according to claim 15, wherein the output of the computer device is a signal or a set of signals that steer the composition of materials that are supplied to the production unit, and wherein the production unit comprises multiple material feeders, wherein an output signal of the computer device steers the quantity of material that is supplied by each feeder to the production unit, wherein each feeder comprises a dedicated material as provided for in the repository.
